(19) United States
(12) Patent Application Publication
(10) Pub. No.: US 2012/0076918 A1
(43) Pub. Date: Mar. 29, 2012

(54) LATENT FINGERPRINT POWDER APPLICATOR AND RELATED METHOD OF USE

(75) Inventors: Eric Aubrey Barton, Wilmington, NC (US); Robert Michael Dameron, Leland, NC (US)

(73) Assignee: TRITECH FORENSICS, Southport, NC (US)

(21) Appl. No.: 12/890,285

(22) Filed: Sep. 24, 2010

(51) Int. Cl. A61B 5/117; A46B 5/04

(52) U.S. Cl. 427/1; 401/7

(57) ABSTRACT

A latent fingerprint applicator apparatus is defined by a sleeve or mitt that is sized to retain the entire hand of a user, the sleeve including a flexible conformable powder application surface that enabling the applicator to be wiped onto a surface of interest to apply powder in order to detect or otherwise identify the presence of latent fingerprints.

### FIELD OF THE INVENTION

The invention relates generally to the field of forensics and more specifically to an apparatus that permits application of powder for purposes of latent fingerprint detection in order to permit identification and a related method for applying fingerprint powder using the apparatus, which is particularly useful in regard to relatively large surface areas, the apparatus being designed and packaged for single (one time) use applications, thereby reducing the possibility of cross contamination at a crime scene.

### BACKGROUND OF THE INVENTION

The hands and feet of persons are naturally oily, due to the presence of eccrine glands, which secrete sweat, a mixture of water, salts and other organic compounds. Sweat adheres to the friction ridges of the fingers, and therefore when a finger touches a surface such as plastic, wood, or glass, an impression (i.e., a fingerprint) will be left behind. The natural oils of the body preserve the fingerprint, which is entirely distinct, from any other individual, thereby establishing a biometric signature of the person which can be subsequently identified.

Latent fingerprints are typically left behind by accident, due to the fact that a careless criminal did not realize that his or her hand left a mark. In other instances, latent fingerprints can be made from substances other than sweat, such as blood, other body fluids, or paint, for example.

When investigators arrive at a crime scene, one of the first things done procedurally after photographing the entire scene is to “dust” the scene for fingerprints. Fingerprint powders are very fine powders are used for this purpose by crime scene investigators and others that are involved in law enforcement. The presence of powder produces a high degree of visual content between the ridge patterns and the surface upon which the fingerprint was left.

Typically, latent detection or “dusting” for fingerprints can be accomplished using a fingerprint wand, brush or similar form of applicator device having a plurality of bristles that permits the adherence of fingerprint dusting powder for depositing onto a surface of interest. Such brush devices include a set of extremely fine fibers that are designed to hold powder and deposit the powder gently on the fingerprint to be revealed, but without rubbing away the often delicate residue of the fingerprint itself. The powder used can be white, (chalk, lanconide, titanium dioxide, or other composition), black (anthracite, charcoal, haddonite black or other composition) or other (aluminum) or other traditionally used material depending on the surface, for example, to which the powder is to be applied. Varying powder formulations can be used, some of which are commonly known and others that are proprietary in nature. While the above-noted technique is effective to reveal the presence of latent fingerprints, it can be time-consuming, inefficient and in fact highly impractical for larger surfaces, e.g., a car body or other similar object.

Another general problem in the field relates to the presence of contamination introduced due to foreign DNA or other matter, which compromises or can render evidentiary information as inconclusive and/or inadmissible to a legal proceeding.

As such, there is a continuing and generalized need in the field of forensics to provide an applicator enabling latent fingerprint detection for subsequent identification, particularly on large surface areas, with the same degree of efficacy that is afforded by presently available brush-type applicator devices that are designed for this purpose.

### SUMMARY OF THE INVENTION

According to one aspect, there is provided a latent fingerprint applicator apparatus, said apparatus comprising an applicator pad having a powder application surface, and means for enabling said apparatus to be maintained in handheld fashion by a user. In one version, the applicator apparatus includes a flexible powder applicator surface that adaptively conforms to the surface area to which the powder is to be applied.

In one version, the applicator apparatus comprises a sleeve or mitt that retains or includes the powder applicator surface. The sleeve according to one version can comprise at least one sheet having edges sealed directly to an applicator pad and defining an interior that is sized to permit the inclusion of a hand of a user of said applicator apparatus. Preferably, the sleeve can accommodate either the left or the right hand of a user.

In one version, the applicator apparatus is designed for single use and or is disposable. According to one embodiment, the applicator pad or surface is made from a section of cotton material, which has preferably been sterilized.

The application pad is preferably sized to permit effective application to a large surface area for purposes of applying powder after which latent fingerprints can be detected from the surface, wherein the powder applicator surface is at least as large as the area defined by the sleeve. The sleeve is preferably integral to the remainder of the applicator apparatus, but can also be releasably attached. In addition, the apparatus is packaged for single (one time) use, thereby reducing the potential incidence of cross contamination.

According to another version, there is provided a latent fingerprint applicator apparatus, said apparatus comprising a sleeve including a powder application side and an opposite side, said sleeve having an interior sized to permit the inclusion of the hand of a user enabling the applicator to be gently wiped onto a surface of interest to apply powder in order to detect or otherwise identify the presence of latent fingerprints.

According to one version, the apparatus can accommodate either the left or right hand of the user. Preferably, the powder applicator surface is made from a suitable material that enables the retention and dispensing of powder, but without damaging any fingerprints that have been left on a particular surface of interest.

According to yet another version, there is provided a method for the detection or identification of latent fingerprints, said method comprising the steps of providing an sleeve having an applicator surface, placing a hand of a user within the interior of the sleeve, applying a quantity of fingerprint powder to the powder applicator surface, and gently moving the applicator pad over a surface of interest using a wiping motion. Preferably, the powder applicator surface is flexible to enable the surface to adaptively conform to the surface to which powder is to be applied and without affecting friction ridges and the ability of the applicator to retain powder.

An advantage obtained using the herein latent fingerprint powder applicator apparatus is that large surface areas can be more easily and readily covered in a faster time and more effectively than using currently defined techniques.

Another advantage is that the applicator apparatus is cost effective and relatively easy to manufacture and use. Moreover, complex training is not a prerequisite to use of the herein described applicator apparatus.

Yet another advantage is that the single-use nature of the herein described apparatus reduces the possibility of cross contamination in regard to a crime scene, thereby helping to insure that evidence obtained at the scene will not be excluded or rendered inadmissible.

These and other features and advantages will become readily apparent from the following Detailed Description, which should be read in conjunction with the accompanying drawings.

### BRIEF DESCRIPTION OF THE DRAWINGS

FIG. 1 is a perspective view of a latent fingerprint powder applicator apparatus in accordance with an first embodiment;

FIG. 2 is a perspective view of the applicator apparatus of FIG. 1, taken from the opposite side and depicting a hand-held portion thereof;

FIG. 3 is a perspective view of the applicator apparatus of FIGS. 1 and 2, illustrating the apparatus in a typical in-use condition;

FIG. 4 is a top plan view of a latent fingerprint applicator apparatus in accordance with a second embodiment;

FIG. 5 is a bottom view of the fingerprint applicator apparatus of FIG. 4;

FIG. 6 is an end view of the fingerprint applicator apparatus of FIGS. 4 and 5; and

FIG. 7 is a perspective view of the fingerprint applicator apparatus of FIGS. 4-6.

### DETAILED DESCRIPTION

The following description herein relates to specific exemplary embodiments relating to a latent fingerprint powder applicator apparatus made in accordance with the present invention. It will be readily apparent that that these embodiments are exemplary and therefore not intended to be overly limiting of the inventive concepts described herein. In addition, several terms are used during the course of the following discussion such as “back”, “front”, “distal”, “proximal” and the like in order to provide a suitable frame of reference with regard to the accompanying drawings. These terms are also not intended to be limiting of the inventive concepts that are taught herein, except where so specifically noted.

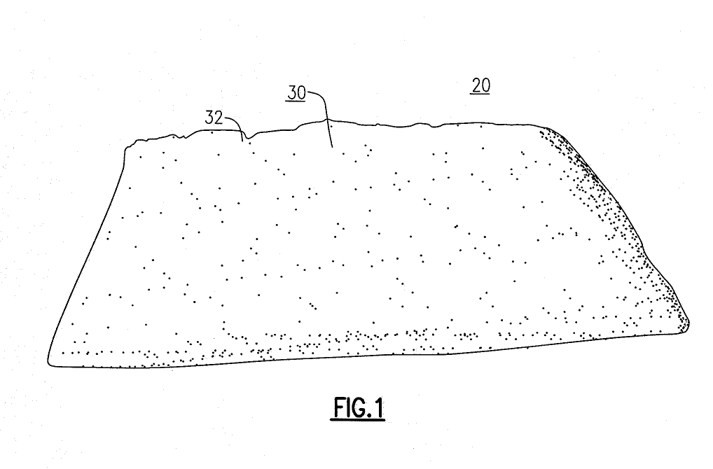

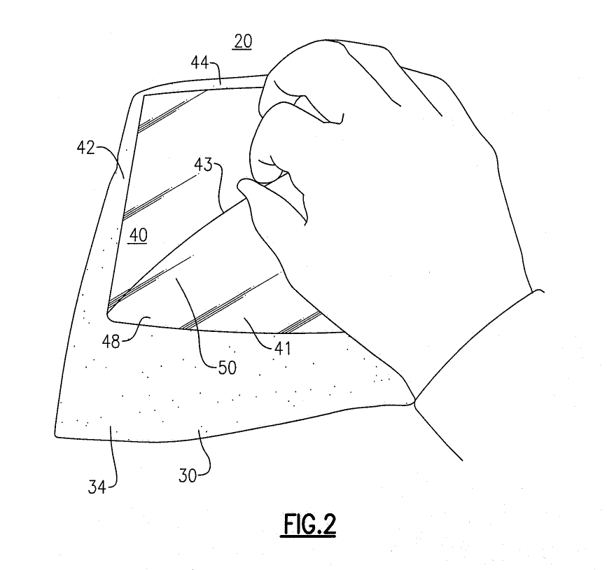

Referring to FIGS. 1 and 2, a latent fingerprint applicator apparatus 20 in accordance with a first exemplary embodiment is formed by a two-part apparatus; namely, an applicator pad 30 and a sleeve 40. The applicator pad 30 is entirely defined by a section of soft non-woven material, such as cotton, which is cut or otherwise formed to a set of defined dimensions. It should be noted that other materials can be used, provided such material possesses the ability to be moved across a surface without affecting friction ridges, as well as the ability to retain powder to permit dispensing thereof, as needed The applicator pad 30 according to this specific embodiment is substantially rectangular in cross section, having a width of approximately 7 inches and a length of approximately 12 inches. It should be noted that the foregoing dimensions, as well as the present aspect (width/length) ratio of approximately 1:2, are intended to be typical examples, provided the proximal surface of the applicator pad 30 is sufficiently large to permit the inclusion of the sleeve 40 or other hand-holdable means, as described below. To that end, the cross section of the applicator pad 30 can assume literally any suitable shape, including circular.

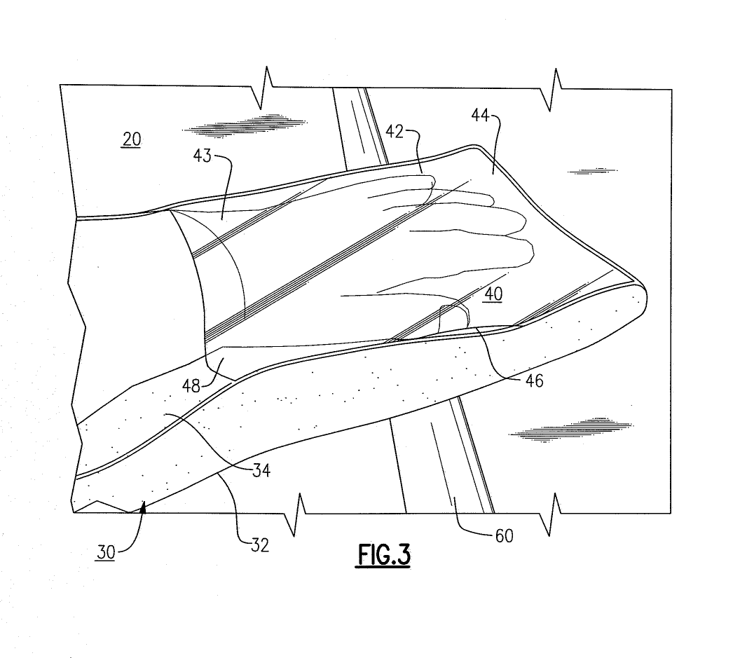

The applicator pad 30 has a distal or “application” side 32 forming the distal or front side of the apparatus 20 and an opposing proximal or support side 34. The sleeve 40 according to this embodiment includes a pair of durable sheets 41, 43, such as polyamide or polyethylene, each sheet having respective perimeter edges 42, 44, 46 that are heat sealed, welded or otherwise affixed together with the exception of an open end 48, forming an interior or pocket that is sized to receive the hand of the user of the apparatus 20. The interior of the sleeve 40 according to this embodiment is sized to substantially fit an entire adult hand therein. One of the plastic sheets 41 of the sleeve 40 is affixed by gluing or other suitable means to the support side 34 of the applicator pad 30. According to this particular version, the applicator pad 30 is sized to substantially conform to that of the sleeve 40. However, the applicator pad 30 can alternatively be sized provided that the sleeve 40 can effectively dust a surface using the apparatus 20, as shown in intended operation by way of example according to FIG. 3.

As shown in FIG. 3, a quantity of a known fingerprint powder such as Carbon Black, manufactured by Tri-Tech Forensics, Inc, is added to the application side 32 of the applicator pad 30 by sprinking a quantity of the powder onto the center of the pad. Numerous powders for this purpose are widely known in the field and selection of same can depend on numerous factors, for example, the color of the surface that is being examined. For example, dark colored powders will better mark a latent fingerprint on a light surface. The cotton material of the herein described applicator pad 30 effectively retains the powder, but still permits its application when the applicator pad 30 is gently wiped across a surface 60 of interest using a reciprocal or sweeping wiping motion in order to detect latent fingerprints from the surface. A gentle wiping motion is all that is necessary to transfer a sufficient amount of powder from the application pad 30 to the surface of interest, wherein the friction ridges of any latent prints are not damaged as a result, readily enabling latent print detection. Either the left or the right hand of a user can be fitted into the interior of the sleeve 40, therefore providing a level of versatility.

According to this particular version, the applicator apparatus 20 is packaged for single (one time) use and is therefore intended to be discarded following use thereof, thereby significantly reducing the potential incidence of cross contamination, as opposed, for example, to reusable fingerprint brushes already known and replete in the field.

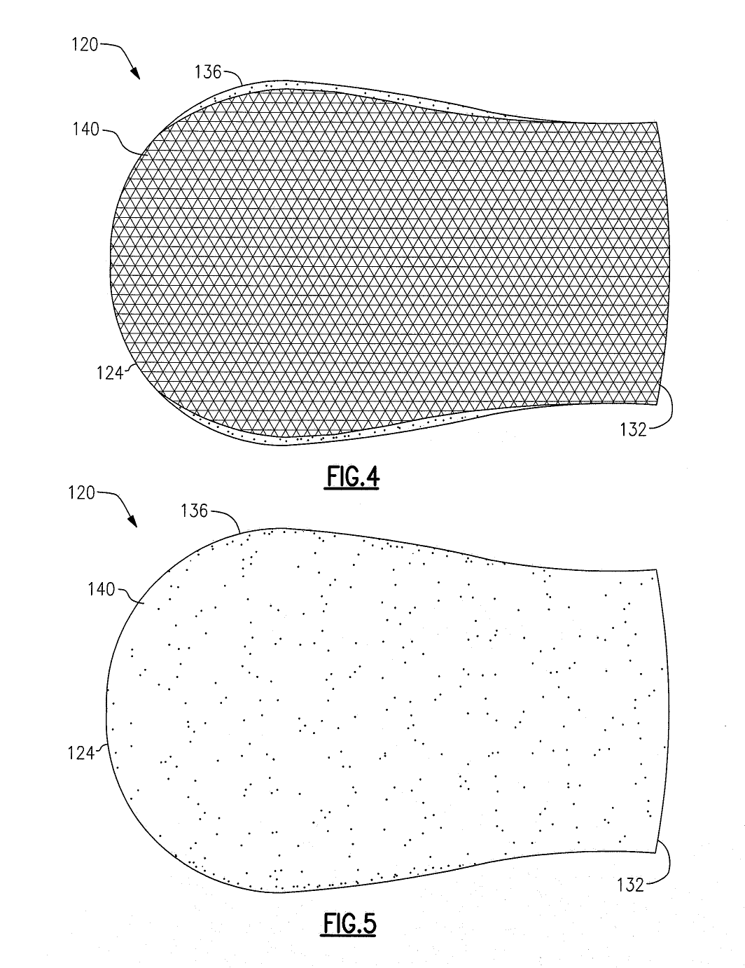

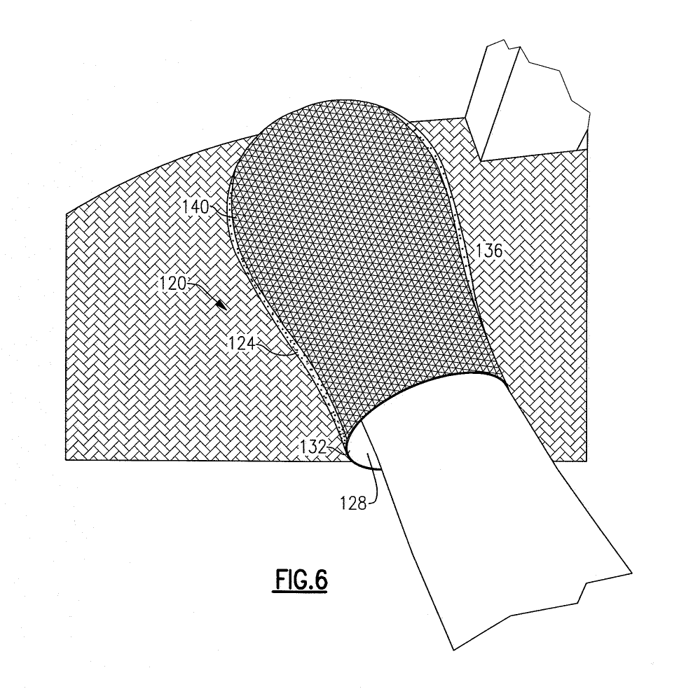

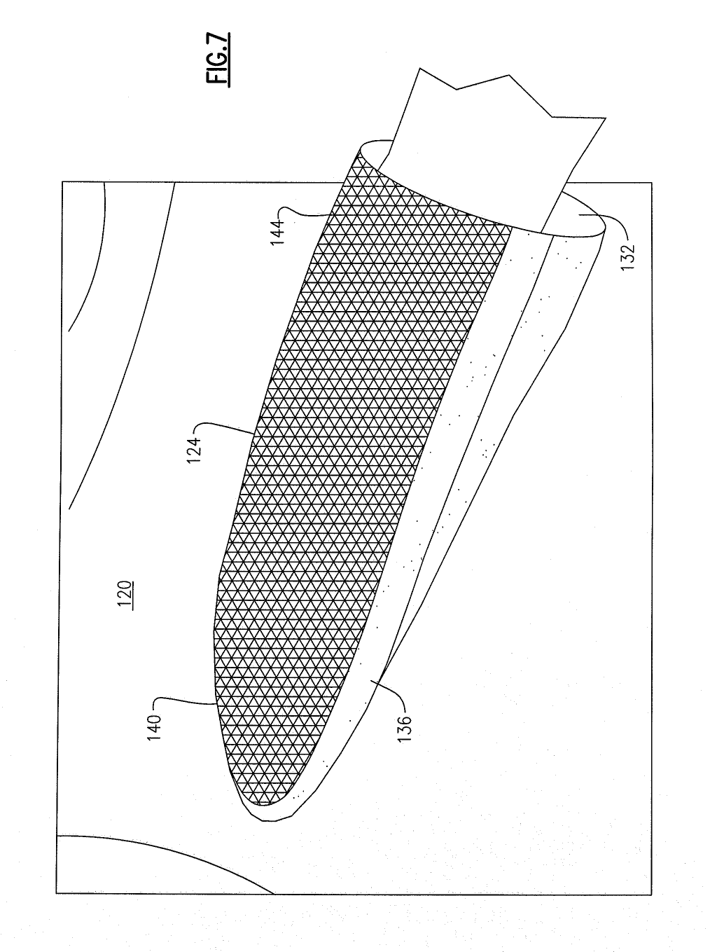

Referring to FIGS. 4-6, a second exemplary embodiment of a latent fingerprint applicator apparatus 120 is herein described. The applicator apparatus 120 according to this version is defined by a applicator sleeve or mitt 124 having an interior 128 sized to receive the entirety of either the right or left hand of a user, as shown specifically in FIG. 6, via an open end 132. According to this embodiment, the mitt 124 is configured with an expanding oval shape extending from a narrowed width at the open end 132 to an expanded curved closed end permitting the entire hand 144, FIG. 7, of the user to be conveniently fitted within the interior of the mitt wherein the fingers and thumb can be fanned outwardly. The applicator mitt 124 according to this embodiment is fabricated from a sheet section 136 of a material, such as cotton, which is stitched along its peripheral edges to a fabric sheet 140 and preferably sterilized to avoid the introduction of foreign DNA and/or other contaminants to the crime scene. As in the latter embodiment, the mitt 124 is preferably pre-packaged for single (one time) use and is discarded after powder has been dispensed. The exterior of the sheet section 136 permits the retention of a quantity of fingerprint dusting powder for depositing of the powder (not shown) similar to that described in the prior embodiment onto a surface area, wherein the flexible mitt 124 substantially conforms to the surface at the crime scene to which the powder is to be applied.

Upon detection of any latent fingerprints, they can be photographed and then compared to those, for example, that are stored in electronic databases such as those found employing the Integrated Automatic Fingerprint Identification System (IAFIS), having storage of over 51 million criminal record subjects and over 1.5 million civil (non-criminal) records.

### Parts List for FIGS. 1-7

20 applicator apparatus
30 applicator pad
32 distal or application side
34 support or proximal side
40 sleeve
41 sheet
42 edge
43 sheet
44 edge
46 edge
48 open end
50 interior or pocket
60 surface
120 applicator apparatus, latent fingerprint
124 mitt
128 interior
132 open end
136 sheet section
140 fabric sheet
144 hand, user

It will be readily apparent that numerous modifications and variations are possible within the intended ambits of the invention, and according to the following claims. For example, the sleeve of the apparatus could be releasably removable, such as by hook and loop fasteners or other suitable means, to a support surface that is attached to the proximal side of the application pad. In another version, the sleeve of the applicator apparatus can be further configured to include provisions for the fingers and thumb for greater stability. Other suitable versions could be contemplated.

1. A latent fingerprint applicator apparatus, said applicator apparatus comprising:
an applicator pad having a powder application surface; and
means for enabling said applicator pad to be hand-held by a user.

2. An apparatus as recited in claim 1, wherein said means for enabling said applicator pad to be hand-held are disposed relative to a surface of said applicator pad opposite said powder application surface.

3. An apparatus as recited in claim 1, wherein at least a portion of said applicator apparatus is disposable.

4. An apparatus as recited in claim 1, wherein said applicator pad is made from cotton.

5. An apparatus as recited in claim 2, wherein said means for enabling said applicator pad to be hand-held comprises a sleeve attached in relation to said surface opposite said powder application surface.

6. An apparatus as recited in claim 5, wherein sleeve comprises at least one sheet having edges sealed directly to the applicator pad and defining an interior that permits the inclusion of a hand of a user of said applicator.

7. An apparatus as recited in claim 6, wherein the sleeve can accommodate either the left or the right hand of a user.

8. An apparatus as recited in claim 6, wherein the applicator pad is sized to accommodate a large surface, and in which the powder application surface is at least as large as the area of said sleeve.

9. A latent fingerprint powder application apparatus, said apparatus comprising:
a mitt including an interior and an open end sized to retain the hand of a user, said mitt having a powder application surface to be wiped onto a surface of interest to apply powder to permit detection of latent fingerprints.

10. An apparatus as recited in claim 9, wherein said mitt is sized to accommodate either the left or right hand of a user thereof.

11. An apparatus as recited in claim 9, wherein said powder application surface of said mitt conforms to the surface area to which powder is applied.

12. A method for the detection of latent fingerprints of large surface areas, said method comprising the steps of:
providing a flexible powder application surface;
placing the hand of a user in a sleeve or mitt retaining said application surface;
applying a quantity of fingerprint powder to said powder application surface; and
wiping said application surface over a surface of interest to deposit powder thereupon for dusting said surface.

13. A method as recited in claim 12, wherein said powder application surface is integral to said sleeve or mitt.

14. A method as recited in claim 12, wherein said sleeve is releasably attachable to an applicator pad having said powder application surface.
